(19) United States
(12) Patent Application Publication
(10) Pub. No.: US 2023/0383972 A1
(43) Pub. Date: Nov. 30, 2023

(54) Air Purifying Device

(71) Applicant: Eric Aaron Langberg, Milford, PA (US)

(72) Inventor: Eric Aaron Langberg, Milford, PA (US)

(21) Appl. No.: 18/205,548

(22) Filed: Jun. 4, 2023

Related U.S. Application Data
(63) Continuation of application No. PCT/US21/61915, filed on Dec. 3, 2021.
(60) Provisional application No. 63/169,980, filed on Apr. 2, 2021.

(51) Int. Cl. F24F 8/80; A61L 9/01; B01D 46/62; B01D 46/00; B01D 46/42; B01D 46/10

(52) U.S. Cl. CPC F24F 8/80; A61L 9/01; B01D 46/645; B01D 46/0049; B01D 46/4263; B01D 46/10; A61L 2209/14; F24F 2221/38; F24F 2221/26; B01D 2279/50; B01D 2273/30

(57) ABSTRACT

An air purifier configured for near-field operation so that purified air is directed toward the head of a user, enabling the user to breathe a higher percentage of purified air versus unpurified ambient air. Additional functions are included to provide enhanced functionality in a use environment, such as on a desk.

### FIELD OF THE INVENTION

The invention generally relates to a device and/or system for removing contaminants from air to improve air quality. More particularly the invention relates to a multipurpose personal use device and system having air purifying capability with enhanced functions.

### BACKGROUND OF THE INVENTION

Air quality and the removal of contaminants is an important consideration for health and safety. Dust, pollen, pet dander, mold spores, and other items can act as allergens, triggering allergies in sensitive people. Further, bacteria, mold spores, and viruses can create illnesses in otherwise healthy persons. Smoke particles and volatile organic compounds (VOCs) can also pose a risk to health. Exposure to various components such as VOCs increases the likelihood of experiencing symptoms of “sick building syndrome”.

Personal breathing apparatuses with a mask and filter are known in in the art, typically called “air purifying respirators” or “powered air purifying respirators”. The user wears a mask with a filter, or a mask with a hose leading to a filtration unit, that draws air through a filter for removal of contaminants. These systems are quite effective for providing purified air to a single user. However, due to social stigma, physical limitations of the device, and difficulty of communication through an impermeable sealed mask, this type of air purification is not practical for general everyday use, day-to-day life, or use in a typical office environment.

Air cleaning devices use ionization, sedimentation chambers, scrubbers, spray chambers, regenerative adsorption, mechanical filtration, and other methods to clean particulates from the air. These devices are intended to process and clean large volumes of air to clean entire rooms, entire buildings, or contaminated air leaving industrial processes. Typical free-standing or portable air purifiers pull in ambient air from around the device and then eject purified air upward into room toward the ceiling to create a circular flow of air within the room. However, if an air processing system or free-standing air purifier is too small for the volume of air in a room cleaning efficiency is degraded as there is not enough air movement to adequately clean the volume of air in the room.

Cleaning the air in large, relatively open, rooms requires large air handling systems, such as building Heating, Ventilation and Air Conditioning (HVAC) systems typically found in commercial buildings. However improperly or poorly maintained HVAC systems can act to further distribute contaminants throughout an indoor environment, rather than remove them. Further, in a large room with an open floor plan, as is commonly found in a commercial office environment with cubicles or open seating arrangements, and sometimes in houses or other buildings, the lack of compartmentalization means there is no way to locally improve the air quality in a given area, such as a cubicle, table or desk area. A typical portable air purifier is too small to effectively clean the air in an open floor plan office or house due to the enormous volume of air.

What is needed is a personal-use system or device with the benefit of a respirator to locally purify air for a user while providing freedom of movement and simplicity of operation. The disclosed apparatus purifies air in a local environment and directs the purified air toward a user to increase the breathing of purified air vs ambient air. The personal use, near-field system represents a departure from typical portable air purifiers since the primary purpose of the disclosed system is to clean the air directly breathed by the user, rather than try to clean all the air in a room.

### SUMMARY OF THE INVENTION

The invented device and system relates to an air purifier configured for operation on a table, desk, counter, or other such elevated surface to be closer to the head of a seated or standing user, to locally improve air quality for a single user or small group of users. The invented air purifier is configured to draw in ambient air, which may include contaminants, from a local region, purify the air by removing a significant portion of the contaminants, and direct the purified air toward a user, or users, such that the user(s) breath(es) in substantially the purified air. Since the purified air is directed where the user is breathing, there is no need to purify a large percentage of the air in a room or space to ensure a user is breathing purified air. This allows a user to be breathing purified air much faster than with a typical air purifier. It also means the air a user is breathing is much less affected by the air quality throughout the rest of the space. The arrangement of the elements of the device provides a higher concentration of purified air around a user's head than ambient air. This results in the user breathing in much more purified air than ambient air, substantially reducing the amount of contaminants the user may breathe. This arrangement provides most of the benefits of a powered air purifying respirator without the need to wear a device or restrict motion during use. Further the invented device or system includes additional functions to increase the utility of the system during use. Additional features and benefits will be described below.

For the purposes of this disclosure, electrical connection shall mean any form of conductor capable of providing a path for flow of electrical current between one or more components.

For the purposes of this disclosure “magnetic material” shall mean any material which exhibits an attraction to a magnet, exhibits magnetism in the presence of a magnetic field, or any material or combination of materials which create a magnetic field, including, but not limited to all types of permanent magnets, ferrous metals, ferrites, ferrates, electromagnets, and other magnetically attractive materials.

The terms “device”, “apparatus”, and “system” are used interchangeably in the specification and it should be understood that these terms may encompass additional elements configured to interface with, be integral with, or be attached to, the disclosed air purifier while still being under the scope of the term.

For the purposes of this disclosure and associated claims, the singular articles “a”, “an”, “the” and other such singular terms are meant to encompass plural versions of the noun to which such articles refer, unless the context specifically dictates otherwise. Where the disclosed system is described as including a single element using the singular articles above, it is understood that the system may incorporate more than one of the referenced element(s) and still be within the scope of this disclosure and claims. For example, if the system description includes “a fan”, it is understood that the system may incorporate more than one fan and still be within the scope of the description or claim.

In an embodiment, an air pressure altering element is used to move air though the system and may comprise a fan, pump, resonator, venturi tube, or other device(s) capable of altering air pressure in the disclosed system.

In an example embodiment, an air purifying device or system is configured to enable placement on a desk, table, counter, or other similar elevated surface such that ambient air is drawn into one or more intake port(s) on at least a portion of the rear of the device and purified outward flowing air is emitted from one or more exit port(s) on at least a portion of the front of the device.

In an example embodiment, an air purifying device or system is configured to enable placement on a desk, table, counter, or other similar elevated surface such that ambient air is drawn into one or more intake port(s) on at least a portion of a side of the device and purified outward flowing air is emitted from one or more exit port(s) on at least a portion of the front of the device.

The air purifying device of any preceding example embodiment or any combination of preceding example embodiments wherein the air purifier comprises a main housing having a front, a rear and at least one side extending from the front to the rear.

The air purifying device of any preceding example embodiment or any combination of preceding example embodiments wherein the air purifier comprises a housing having a front, a rear, and at least two sides extending from the front to the rear and wherein at least one side forms an angle with the front of less than 90 degrees.

The air purifying device of any preceding example embodiment or any combination of preceding example embodiments wherein outward flowing air is directed toward the head of a person or persons in a user position relative to the device.

The air purifying device of any preceding example embodiment or any combination of preceding example embodiments, wherein the air purifying device is configured to resemble a common picture frame. Since a desk is typically below the head of a user, the front of the air purifying device is angled backward to direct the outward flowing air toward the head of a user or users in a user position relative to the device.

The air purifying device of any preceding example embodiment or any combination of preceding example embodiments, wherein the air purifying device is configured with a desk lamp including a lighting means to illuminate an area or region of the desk.

The air purifying device of any preceding example embodiment or any combination of preceding example embodiments, wherein the air purifying device is configured as a desktop monitor stand having a top surface oriented substantially parallel to the desk and wherein the top surface is configured to support a computer monitor and wherein the top surface elevates the computer monitor above the surface of the desk by the height of the air purifying device.

The air purifying device of any preceding example embodiment or any combination of preceding example embodiments, wherein the air purifying device is configured as a desktop monitor stand having a top surface oriented substantially parallel to the desk and wherein the top surface is configured to support a computer monitor and wherein the top surface elevates the computer monitor above the surface of the desk by one or more predefined distances.

The air purifying device of any preceding example embodiment or any combination of preceding example embodiments, wherein the air purifying device is configured with a charging station to provide charging energy to one or more rechargeable devices, such as, but not limited to portable electronics, computing devices and telecommunications devices.

The air purifying device of any preceding example embodiment or any combination of preceding example embodiments, wherein the air purifying device is configured with a wireless charging station and holder for one or more rechargeable devices, such as, but not limited to, cell phone, tablet, watch, radio, speaker system, or other personal electronics device.

The air purifying device of any preceding example embodiment or any combination of preceding example embodiments, wherein the air purifying device is configured with a speaker system having means for amplifying and producing sound from one or more built-in speakers.

The air purifying device of any preceding example embodiment or any combination of preceding example embodiments, wherein the air purifying device is configured with a desktop communications device with means, such as one or more speakers, for reproducing audio, and means, such as one or more microphone, for receiving audio from the use environment, for example speech from a user.

The air purifying device of any preceding example embodiment or any combination of preceding example embodiments, wherein the air purifying device comprises a rear compartment, a central compartment, and a front chamber.

The air purifying device of any preceding example embodiment or any combination of preceding example embodiments, wherein the air purifying device comprises a filter or filter assembly configured to fit into a chamber or compartment, and an air pressure altering element, such as a fan, configured to create a negative air pressure on a first side of the air pressure altering element and a positive air pressure on a second side of the air pressure altering element.

The air purifying device of any preceding example embodiment or any combination of preceding example embodiments, wherein the air pressure altering element is positioned within the air purifying system such that during operation, the air pressure altering element creates a negative pressure on an inward face of the filter assembly, thereby drawing ambient air through the filter assembly and into the air purifying device.

The air purifying device of any preceding example embodiment or any combination of preceding example embodiments, wherein the air pressure altering element is positioned within the system such that during operation, the air pressure altering element draws in ambient air and creates a positive pressure on an input face of the filter.

The air purifying device of any preceding example embodiment or any combination of preceding example embodiments, wherein the air purifying device comprises a filter assembly configured to fit into a housing, a fan configured to draw in ambient air through an air intake region and force the ambient air through the filter to create filtered air. The filtered air is routed through the housing and configured to exit the front of the housing through an exit port or exit ports.

The air purifying device of any preceding example embodiment or any combination of preceding example embodiments, wherein the air purifying device includes a front baffle to help control air currents of the outward flowing air.

The air purifying device of any preceding example embodiment or any combination of preceding example embodiments, wherein the front housing component includes features on its interior side to help control air currents of the outward flowing air.

The air purifying device of any preceding example embodiment or any combination of preceding example embodiments, wherein a front baffle is integral with a front housing component.

The air purifying device of any preceding example embodiment or any combination of preceding example embodiments, wherein a front baffle is removably attached to a front housing component.

The air purifying device of any preceding example embodiment or any combination of preceding example embodiments, wherein the front baffle incorporates a structure to secure a photograph, calendar, document, printed item, or other visual element.

The air purifying device of any preceding example embodiment or any combination of preceding example embodiments, having a movable outer element on the front which is movable from a first closed position to at least a second open position, and where the movable outer element is configured to support a personal electronics device in the open position.

Securing provisions may be included on the front or extending from one or more sides for supporting an external device such as a cell phone or tablet, etc. Resting support may be built-in or removable via engagement features. For example, a side of the picture frame may flip out to provide the resting support in a first position and flip back to provide a closed frame in a second position. Slots in the front baffle may be included for inserting a bracket as a resting support. A resting support may be permanently attached to or integral with the front baffle by providing a lip on one or more edges of the display area or sides of the front baffle.

The air purifying device of any preceding example embodiment or any combination of preceding example embodiments, wherein the front baffle comprises a graphic display for the display of static or moving images such as photographs, graphics, movies, television images, video, digital streaming, etc.

The air purifying device of any preceding example embodiment or any combination of preceding example embodiments, wherein the front baffle comprises an electronic display linked to an external device or system such that the display is capable of displaying information, content, or images received from the external device or system.

The air purifying device of any preceding example embodiment or any combination of preceding example embodiments wherein an electronic display includes a touch-screen for changing content in response to touches from a user.

The air purifying device of any preceding example embodiment or any combination of preceding example embodiments wherein the display region display includes user interface controls for altering system operating in response to actions from a user.

The air purifying device of any preceding example embodiment or any combination of preceding example embodiments, wherein the air purifying device comprises a power source and control circuit.

The air purifying device of any preceding example embodiment or any combination of preceding example embodiments, further comprising a user-controlled switch for controlling power from the power source to at least a fan.

The air purifying device of any preceding example embodiment or any combination of preceding example embodiments, further comprising an illumination means.

The air purifying device of any preceding example embodiment or any combination of preceding example embodiments, wherein the front baffle is transparent or translucent and the illumination means is configured to illuminate the front baffle or the perimeter of the front panel.

The air purifying device of any preceding example embodiment or any combination of preceding example embodiments, wherein the air purifying device includes a movable element configured to enable aiming of outflowing air.

The air purifying device of any preceding example embodiment or any combination of preceding example embodiments, wherein the air purifying device includes a movable element configured to enable directing, aiming or focusing of light from a lighting means.

The air purifying device of any preceding example embodiment or any combination of preceding example embodiments, wherein the air purifying device further comprises one or more elements configured to alter the condition of the outward flowing air relative to the ambient air thereby changing the condition of the temperature, humidity, scent, smell, level of pollutants, ionization level, or concentration of gases, such as oxygen level.

The air purifying device of any preceding example embodiment or any combination of preceding example embodiments, wherein the front chamber, central compartment, or main chamber is configured as a conditioning chamber to create conditioned air.

The air purifying device of any preceding example embodiment or any combination of preceding example embodiments, wherein the air purifying device further comprises a temperature altering element configured to change the air temperature of the outward flowing air relative to the ambient air.

The air purifying device of any preceding example embodiment or any combination of preceding example embodiments, wherein the air purifying device includes a high-efficiency particulate air (HEPA) filter, also sometimes referred to as a high-efficiency particulate absorbing filter or high-efficiency particulate arrest filter.

The air purifying device of any preceding example embodiment or any combination of preceding example embodiments, wherein the air purifying device further comprises an ultraviolet (UV) light for destroying microbes and wherein air is passed through UV sanitizing chamber before exiting the air purifier.

The air purifying device of any preceding example embodiment or any combination of preceding example embodiments, further comprising an ultraviolet (UV) light and wherein the UV light is arranged to destroy microbes trapped in the filter.

The air purifying device of any preceding example embodiment or any combination of preceding example embodiments, further comprising an ultraviolet (UV) light integral with a filter assembly.

The air purifying device of any preceding example embodiment or any combination of preceding example embodiments, wherein the air purifying device further comprises an ionization element to alter the concentration of negative or positive ions in the outflowing air relative to the ambient air.

The air purifying device of any preceding example embodiment or any combination of preceding example embodiments, wherein the air purifying device further comprises an ionization element to alter the concentration of negative or positive ions in the inward flowing air relative to the ambient air before passing the inward flowing air through the filter.

The air purifying device of any preceding example embodiment or any combination of preceding example embodiments, further comprising an ionization element integral with a filter assembly.

The air purifying device of any preceding example embodiment or any combination of preceding example embodiments, wherein the air purifying device further comprises a water holding tank.

The air purifying device of any preceding example embodiment or any combination of preceding example embodiments, wherein the air purifying device comprises a humidity altering element configured to extract water from the flowing air and divert the water to the water holding tank to create conditioned air with lower humidity than the ambient air.

The air purifying device of any preceding example embodiment or any combination of preceding example embodiments, wherein the air purifying device further comprises a humidity altering element configured to introduce water from the holding tank to the flowing air to create conditioned air with higher humidity than the ambient air.

The air purifying system of any preceding example embodiment or any combination of preceding example embodiments, wherein the air flow through the system is in the range of four (4) cubic feet per minute to twenty (20) cubic feet per minute.

The air purifying device of any preceding example embodiment or any combination of preceding example embodiments, further comprising a battery configured to supply power to the system.

The air purifying device of any preceding example embodiment or any combination of preceding example embodiments, wherein the sound pressure level (SPL) of the air and fan noise is below 60 dBA at a distance of one-half (½) meter.

The air purifying device of any preceding example embodiment or any combination of preceding example embodiments, wherein the front chamber or baffle is configured to reduce the sound pressure level of the outward flowing air.

The air purifying device of any preceding example embodiment or any combination of preceding example embodiments, wherein the front chamber further comprises noise reducing elements.

The air purifying device of any preceding example embodiment or any combination of preceding example embodiments, wherein the front chamber houses one or more panels configured to guide air flow from the fan toward the output port(s).

The air purifying device of any preceding example embodiment or any combination of preceding example embodiments, wherein the front surface is configured to receive attachments.

The air purifying device of any preceding example embodiment or any combination of preceding example embodiments, wherein attachments are removably attached to the front surface engagement features.

The air purifying device of any preceding example embodiment or any combination of preceding example embodiments, wherein engagement features include hook and loop fasteners, magnet(s), interlocking mechanical connectors, clamps, slots, tape, removable glue, electrostatic adhesion, or other removable engagement means.

The air purifying device of any preceding example embodiment or any combination of preceding example embodiments, wherein the front surface comprises a removable structure.

The air purifying system of any preceding example embodiment or any combination of preceding example embodiments, wherein the front surface comprises a removable front structure and further comprising additional interchangeable front structures. Additional front structures may comprise, singly or in any combination, a picture frame, a dry erase board, a pincushion, a corkboard, a document holder, a chalkboard, a magnetic material, an artwork.

The air purifying device of any preceding example embodiment or any combination of preceding example embodiments, further comprising a receiver for attaching elements to the air purifier. In an embodiment the receiver comprises a threaded insert.

The air purifying device of any preceding example embodiment or any combination of preceding example embodiments, further comprising mounting provision for securing the air purifier to an external mounting support, attached to pole, music stand, microphone stand, riser next to cash register, hand rail, mounting bracket, etc.

### BRIEF DESCRIPTION OF THE DRAWINGS

Having thus described the disclosure in general terms, reference will now be made to the accompanying drawings, which are not necessarily drawn to scale, and wherein:

FIG. 1A, FIG. 1B and FIG. 1C illustrate a front view, a rear view, and a side view, respectively of an air purifying device according to one example embodiment of the present disclosure;

FIG. 2A and FIG. 2B illustrate a front exploded view and a rear exploded view, respectively, of an air purifying device according the one example embodiment of the present disclosure;

FIG. 3 illustrates a block diagram of a control circuit for an air purifying device according to an example embodiment of the present disclosure;

FIG. 4 illustrates an air purifying system configured as a display device according to one embodiment of the present disclosure;

FIG. 5A and FIG. 5B illustrate a side view and a side cutaway view, respectively, of an air purifying system according to one example embodiment of the present disclosure;

FIG. 6 illustrates a side cutaway view of an air purifying system including a condition altering element according to one example embodiment of the present disclosure;

FIG. 7 illustrates a side cutaway view of an air purifying system including a condition altering element according to one example embodiment of the present disclosure;

FIG. 8A and FIG. 8B illustrate an example embodiment configured as a stand for an external display device;

FIG. 9 illustrates an exploded side view of an example embodiment with included air duct system;

FIG. 10 illustrates a side cutaway view of a portion of an embodiment with air duct system with rear housing and other components removed;

FIG. 11A and FIG. 11B illustrate separated views of various elements of an embodiment with included air duct;

FIG. 12A, FIG. 12B, FIG. 12C, FIG. 12D, FIG. 12E, and FIG. 12F show various configurations of an embodiment of the front baffle with removable elements;

FIG. 12G illustrates an embodiment of a front baffle with securing means for a document or visual element;

FIG. 13 illustrates an embodiment of a front baffle with a retaining feature;

FIG. 14 illustrates an embodiment of a front panel for holding multiple items.

FIG. 15A and FIG. 15B illustrate embodiments of an air purifier system with electronic display integral with a front baffle and provision for connection to external devices.

### DETAILED DESCRIPTION

The current disclosure will now be described more fully hereinafter with reference to exemplary embodiments thereof. These exemplary embodiments are described so that this disclosure will be thorough and complete, will fully convey the scope of the disclosure to those skilled in the art, and satisfy applicable legal requirements. Indeed, the disclosure may be embodied in many different forms and should not be construed as limited to the embodiments set forth herein. As used in the specification, and in the appended claims, the singular forms “a”, “an”, “the”, include plural variations unless the context clearly dictates otherwise.

An air purifying device is disclosed herein, which may be configured in a variety of forms. The air purifying device includes components to remove pollutants from the ambient air in a local region of a room or building.

In an embodiment the air purifying device includes a housing which may be made from plastic, metal, wood, glass, or other suitable materials. The housing may be formed as an integral structure, such as by molding, machining, or additive manufacturing, or may be constructed from multiple components. In embodiments including a display feature, housing includes at least a rear and a front. Other shapes, such as cylindrical, spherical, conical, pyramidal are contemplated where “front” and “rear” are not readily determined by the shape of the housing, but by the location of certain features, such as air exit ports. As used in this disclosure, “front” generally means toward or proximal to a user, user position or target area, and “rear” generally means away from or distal to a user, user position or target area.

Air purifying device includes an air intake region with one or more air inlet ports on at least a portion of the housing for accepting inward flowing air. Air inlet ports may be formed as discrete holes or perforations in the housing or the air intake region may comprise a mesh, foam, or other air-permeable material with integral air inlet ports comprising openings or spaces in the air-permeable material. It is preferable to have a total surface area of air intake being larger than the total surface area of air exiting from the system. The housing may comprise one or more removable elements comprising air intake region, such as a removable screen for serving as a prefilter, air intake region, and securing means for a removable or replaceable filter. Air inlet ports may be configured on the rear of the device or on the side of the device. The air intake region may be part of an openable or removable component, such as a cover.

Air purifying device includes an air discharge region for outward flowing air on at least a portion of the front of the device. The air discharge region includes one or more exit ports. In an embodiment, the exit ports are arranged around the periphery of a front face of the device.

As outward flowing air is discharged from the front of the device toward a user in a user position, the outward flowing air creates a stream of moving air which expands to form an increasingly larger cross-sectional area, or wave front, as the stream of air moves further away from the device. The outward flowing air creates a generally tapered, conical or wedge-shaped stream of moving air, with a wave front which may be roughly circular or rectangular in cross-section. The area of the wave front of the conical outward flowing air is controlled to create a high concentration of purified air across the wave front. As the wave front passes around the head of a user in a user position, the air around the head of the user is substantially all purified air, such that the user breathes in purified air with every breath while the user's head is in the user position. The area of the wave front may be controlled to create a larger or smaller wave front at a user position by altering the angle of the exit ports relative to the front of the air purifying device. The size of the wave front and concentration of purifier air to ambient air may also be affected by the speed of air flow through the system or the selective activation of exit ports.

In an embodiment, the outward flowing air is controlled to create stream of moving air with a cross-sectional area of at least seventy-five square inches at a user position distance of between eighteen inches to thirty-six inches away from the front of the device.

Air purifying device includes a filter or filter assembly. The filter is configured to mechanically capture contaminants as air is passed through the filter. The filter is arranged with an input face being a first face, side, plane, or surface oriented toward an input region of the air purifier. The filter has an output face being a second face, side, plane, or surface oriented toward the output region of the air purifier. During use, the input face will receive ambient air, which will flow through the filter to the output face, resulting in flow of purified air from the output face. The filter may comprise a single filter media component, or may be an assembly employing more than one component of filter media or more than one type of filter media. In an embodiment, the filter assembly comprises multiple layers and is configured as a HEPA filter. In an embodiment, the filter is located in a chamber with a screen exterior of the filter to protect or secure the filter while allowing the ingress of ambient air. In an embodiment the filter assembly includes an activated charcoal layer and a screen layer integral with the filter assembly. The filter assembly may further include an element for providing ultraviolet (UV) light, an ionization element, individually or in combination, such that the filter assembly incudes more than one air purification method.

In an embodiment the air purifying device includes an element for increasing the air pressure such that air exits the device at a higher pressure than enters the device. The element for increasing the air pressure may comprise a fan, pump, blower, or other electromechanical means for increasing air pressure in response to an electrical signal. In some embodiments, the system may use fan with rotary vanes, commonly referred to as a “squirrel cage” fan. In some embodiments the element for increasing the air pressure may employ a Venturi arrangement whereby more air is drawn into the system than passes directly over the vanes of a fan.

The filter and air pressure increasing element may be arranged so that ambient air is drawn into and through the filter by reducing the air pressure on the output face of the filter relative to the input face of the filter. In an embodiment where the air pressure increasing element is a fan, the air pressure on the output face may be reduced by having the fan arranged such that the input air of the fan comes from the output face of the filter. The output air of the fan is then directed to the output region of the air purifier.

In an embodiment, the fan is arranged so that the input air to the fan comprises the ambient air and the output air of the fan creates a higher pressure on the input face of the filter, pushing the air through the filter to the output face of the filter.

Air purifying device may include a stand, bracket, foot, base, or other stabilizing element for positioning the device at a preferred angle. The stabilizing element may be fixed, movable, or adjustable. The stabilizing element may be detachable and attachable for orienting the device in various positions, such as portrait or landscape, or mounting on the wall of a cubicle. In portrait orientation, the longest side of the device would be oriented substantially perpendicular to the ground or resting surface. In landscape orientation the longest side of the device would be oriented substantially parallel to the ground or resting surface. The stabilizing element may be configurable in more than one position to alter the angle the front of the air purifier relative to a surface on which it rests. The stabilizing element may be continuously variable or able to be secured in one or more preset positions.

The air purifying device includes a main chamber or front chamber for high pressure air. A front panel encloses the front of the main chamber. The front panel may be made from an opaque material such as plastic, metal, wood or other suitable material, or may be made from a translucent or transparent material to allow the transmission of light through the front panel. Main chamber leads to the air discharge region so that high pressure air is expelled from the device through one or more exit port(s). The angle of the exit ports relative to the desktop aims the outward flowing air toward the head of a user. The angle may be controlled by the shape of the housing, the angle of the front of the device relative to the surface upon which it's resting, or by the angle of the exit ports relative to the resting surface. Exit ports may include movable element or elements, such as vanes, to direct the outward flowing air toward the head of a user.

In an embodiment, the air purifying device includes a display region for holding a visual element such as a calendar, photograph, painting, print, decoration nor other visual item. The display region may be included as part of a front baffle, or may be built into a front housing component of the system. The element comprising the display region may be made from an opaque material, a translucent material, or transparent material. When the display region comprises a translucent or transparent material, the visual element may be illuminated by light passing through the display region from one or more lighting elements positioned behind the display region. Alternatively, the display region may include a graphic display capable of displaying digital pictures, graphics, movies, or other electronic content. In an embodiment, the front panel or front housing is configured to retain a cell phone, tablet, video monitor, computer monitor, or other personal media device with integral graphic display.

The display region may comprise a recessed portion of a front panel or front baffle with the recessed portion configured to receive a photo or other visual element. The display region may further comprise one or more notch, slot or opening for sliding the visual element into the recessed region.

In an embodiment, the display region comprises a recessed portion configured to receive and retain at least a portion of an external device such as a portable media device or personal computing device with integral graphic display including, but not limited to, a cell phone, tablet, computer monitor, etc. The external device may be supported on a single side, for example at a bottom side closest to a resting surface of the air purifier, or may be secured in more than one location. In an embodiment, the external device is generally rectangular and is supported at least as two corners of the device. Support my be accomplished using clips, brackets, hooks, detachable fasteners, flexible elements such as elastic bands or other flexible element(s), magnets, framing pieces, or other securing means, either singly or in any combination thereof.

The air purifying device incudes a control circuit in electrical communication with the power source and the air pressure increasing element to control operation of the air pressure increasing element and any other optional electrical features, such as, but not limited to, user interface electronics, lighting elements, other optional features as described in this specification. The control circuit may comprise one or more user control(s) to receive input from a user and control such things as on/off, fan speed, temperature, humidity, or any other controllable variables. The control circuit may be configured to provide for more than one fan speed setting or may provide for continuously variable control of fan speed. In an embodiment, the control circuit includes at least one setting whereby control of the air pressure increasing element results in an air flow rate through the system of between four (4) cubic feet per minute to twenty (20) cubic feet per minute.

The control circuit may also include user interface feedback elements such as lights, numeric displays, graphic displays, or other such features to inform a user about the operating status of the system. The control circuit may include a clock to inform user of the time and/or may include a clock and/or timer which may be set by a user for predetermined actions such as turning the system on or off at predetermined times, running the system for a set time period, initiating maintenance actions, etc.

In an embodiment, the air purifying device includes additional features to increase its utility during use. Incoming power, or internal battery power if so equipped, may be routed to provide power for charging one or more external rechargeable device(s). For example, and without limitation, these external rechargeable devices may include one or more laptop, cell phone, tablet, camera, wireless computer mouse, audio player, watch, etc. Charging power may delivered to the rechargeable device via wired connection or via wireless means, such as, but not limited to, inductive or capacitive charging means. In an embodiment, the air purifier includes one or more output connections for providing charging power. Output connections may include any variety of electrical connectors, including industry standard or proprietary connectors such as various iterations of universal serial bus (USB) connectors (USB-A, USB-B, USB-C, USB Mini, USB micro, etc) “lightning” connectors from Apple, ethernet, etc. In an embodiment, the air purifier includes a USB hub including a USB hub controller and appropriate connectors for providing USB data connection between a USB host device or USB master device and USB client devices or USB peripheral devices. The USB hub may employ USB On-the-Go (OTG). Power may be supplied from the air purifier to create a powered hub and/or to power USB OTG devices.

Various exemplary embodiments will be used in describing the invention for ease of explanation of features, without limiting the invention to such exemplary embodiments. Various elements and features in the descriptions below may be combined in any manner to create new embodiments within the scope of the invention. For ease of explanation, basic terms will be used to describe the embodiments, with the understanding that those terms encompass the equivalent variations described in this specification.

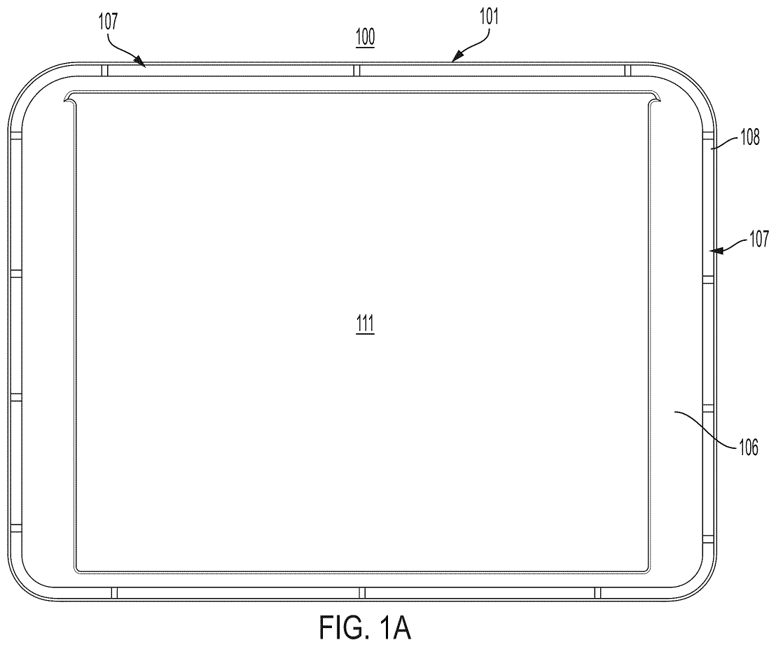

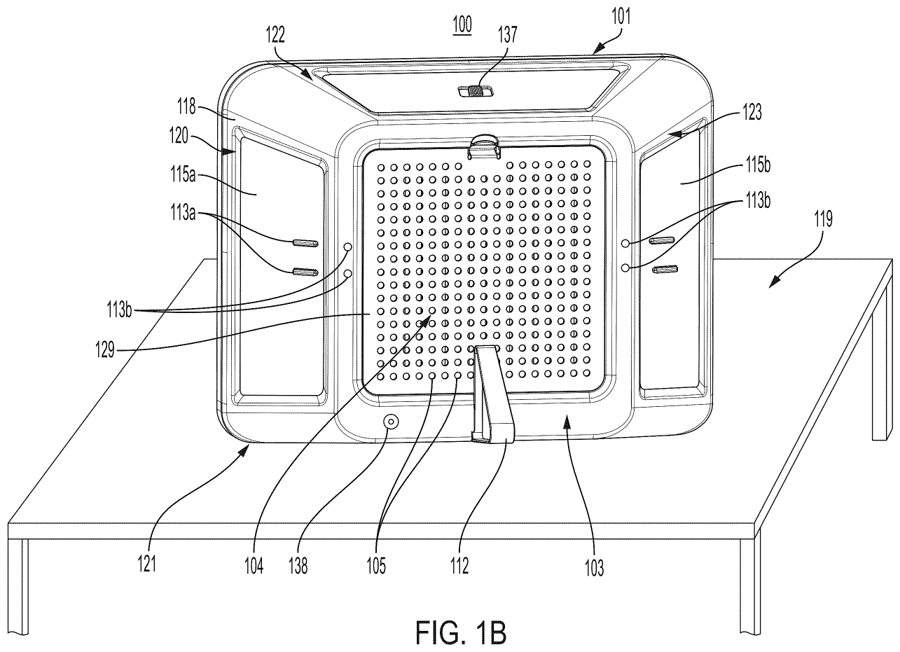

Referring to FIGS. 1A, 1B and 1C, an air purifying device 100 is shown configured as a desktop display system. The system includes housing 101 having a front 102, rear 103, and sides 120, 121, 122, 123. Sides 120-123, may each comprise one or more side surfaces 115, 118. At least one side 120-123 forms angle A with front 102 and is configured to serve as a contact side for supporting the device 100 on a desk or other resting surface 119. Stand 112 may be removably attached to one or more housing portions via retaining features 113a, 113b to support the device in one or more positions. For example, stand 112 may be engaged with retaining features 113a on a first side surface 115 to support device 100 in an upright, or portrait, orientation; or stand 112 may be engaged with retaining features (not shown) on side 121 or rear 103 to support device 100 in a sideways, or landscape, orientation. Housing 101 may also be configured with each side surface 118 forming a different acute angle A relative to front 102, and with stand 112 configured to support device 100 in combination with any side surface, for example 118, thereby providing a means to vary the angle A of front 102 relative to a resting surface 119 just by changing which side surface, for example 118, is acting as the contact surface with the resting surface 119. Retaining features 113b may be located on rear 103 in addition to, or in place of, retaining features 113a on one or more side surface, for example 118. Side surface 115 may be recessed relative to side surface 118 so that retaining features 113a do not extend above the plane of side surface 118.

Air intake region 104 is disposed on at least a portion of rear 103. Air intake region 104 may be formed integral with housing 101 or may be formed via separate panel or screen removably attached to housing 101. Air intake region 104 comprises air inlet ports 105 for receiving ambient air into the device. One or more user control(s) 137 may be provided for a user to control operation of the system.

Air purifying device 100 further includes a front baffle 106 with display region 111, air discharge region 107 comprising one or more exit port(s) 108. Display region 111 may be configured to accept and retain a visual element 109, which may be secured behind optional transparent pane 110, which are shown unsecured from front baffle 106. Transparent pane 110 may be made from clear plastic, glass, or other transparent substance to secure and protect visual element 109. Air discharge region 107 is arranged peripheral to front baffle 106 and may encompass the entire periphery of front baffle 106 or portions of front baffle 106. Similarly, exit port 108 may encompass the entire periphery of front baffle 106 as single exit port, or may comprise one or more smaller exit ports disposed along portions of front baffle 106. Display region 111 may comprise a built-in or insertable graphic display or separate device with an integral display. While FIGS. 1A, 1B and 1C depict a housing having a substantially rectangular front 102, it is understood and expected that the shape could be any shape to meet cosmetic or industrial design goals. In such alternate shapes, the features described above would be altered, or in some cases eliminated, to accomplish the goals.

Air exiting via exit port 108 leaves device 100 in a direction substantially perpendicular to front baffle 106. As angle A is decreased from 90 degrees, air exiting device 100 exits at an increasingly upward angle relative to resting surface 119. From simple geometry it can be observed that the upward angle may be found by subtracting angle A from 90 degrees. In other words, the upward angle and angle A are complementary angles.

Power to the system may be supplied through power connection 138, which may be permanently attached or removably coupled, and which may be configured to receive power from an alternating current (AC) or direction current (DC) source. If an optional rechargeable battery (not shown) is included in system 100, power received at power connection 138 may be used to power the system and/or recharge the battery.

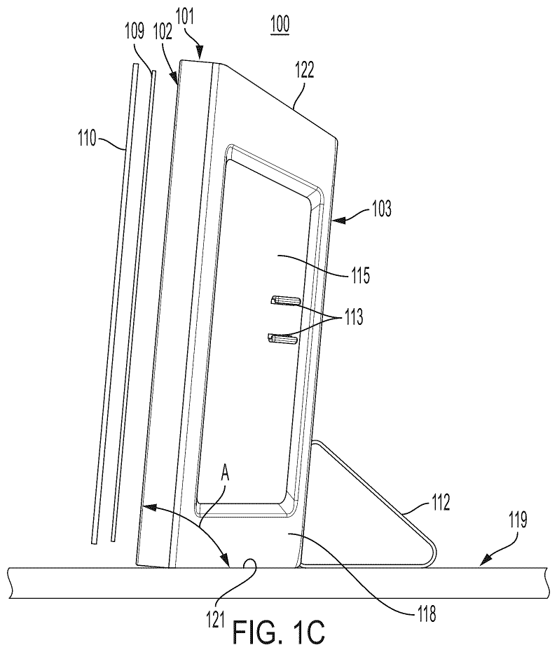

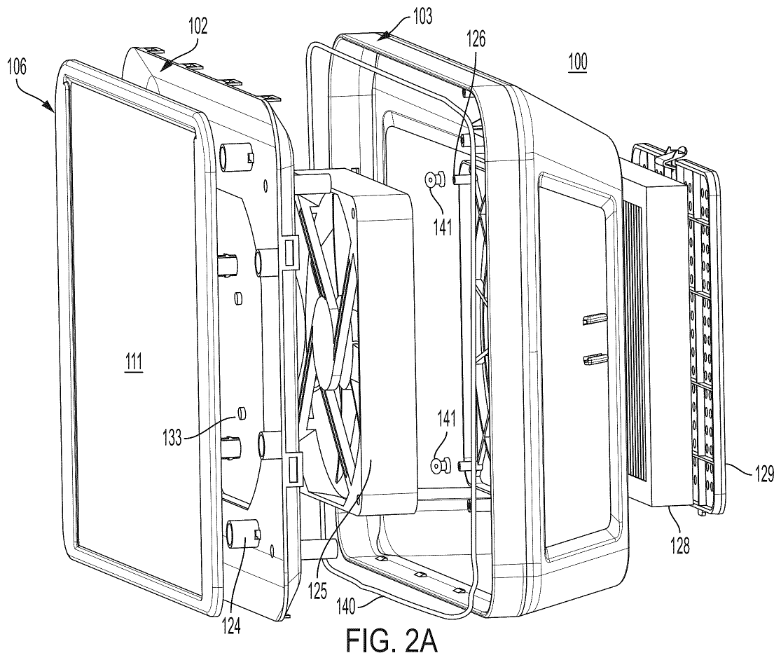

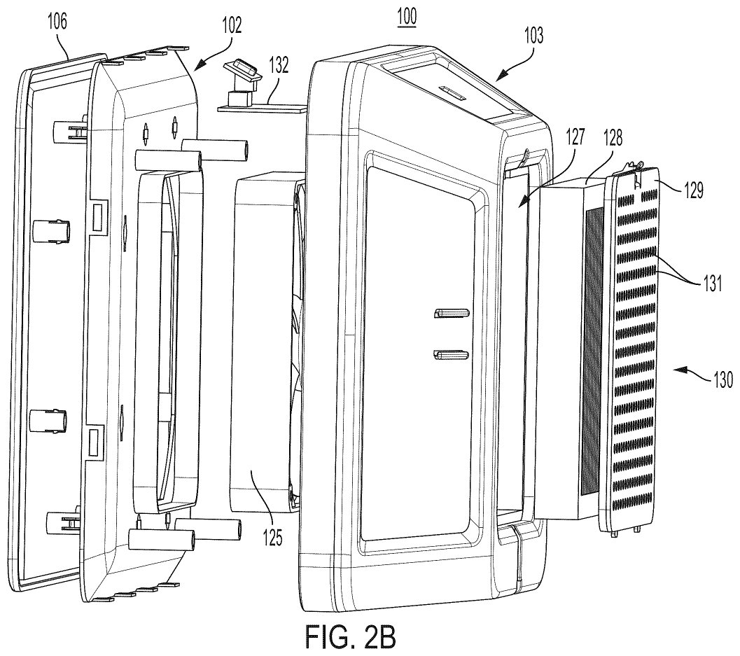

FIG. 2A and FIG. 2B shows a front exploded view and a rear exploded view, respectively, of the system embodiment in FIGS. 1A, 1B, and 1C. Front housing component 102 interfaces with rear housing component 103 to form a main chamber (not shown). Front housing component 102 includes mounting features 124 for securing front baffle 106, which includes display region 111. Fan 125 is secured to fan mounting features 126 in rear housing 103. To reduce vibration within the system, resilient damping material 140 may be employed at the respective joining surfaces of front housing component 102 and rear housing component 103. Vibration reduction may also be included in mounting of fan 125 via resilient damping material (not shown) between contact areas of the housing of fan 125 and rear housing component 103, by the inclusion of resilient mounting elements 141 configured to reduce direct coupling of vibration of fan housing of fan 125 to fan mounting features 126, or inclusion of both resilient damping material between fan housing of fan 125 and rear housing component 103, and resilient mounting elements 141. Rear housing component 103 includes rear chamber 127 for securing filter 128. Filter screen 129, with air intake region 130 covers rear chamber 127 and filter 128. Air intake region 130 includes air inlet ports 131 for accepting ambient air into the device.

Control circuit board assembly 132 may be secured to front housing component 102 or rear housing component 103. Optional lighting element 133 may be positioned behind front baffle 106 to illuminate around the periphery of front baffle 106. Lighting element 133 is in electrical connection with control circuit board assembly 132 so that illumination of lighting element 133 is controlled by control circuit board assembly 132. Optional lighting element 133 may also be used to shine through a transparent or translucent front baffle 106. Multiple lighting elements 133 may be used to increase light output or to provide a more consistent or even illumination.

Stabilizing element 112 is configured to be detachable and movable, engaging with retaining features 113. Retaining features 113 may be located on various sides of housing

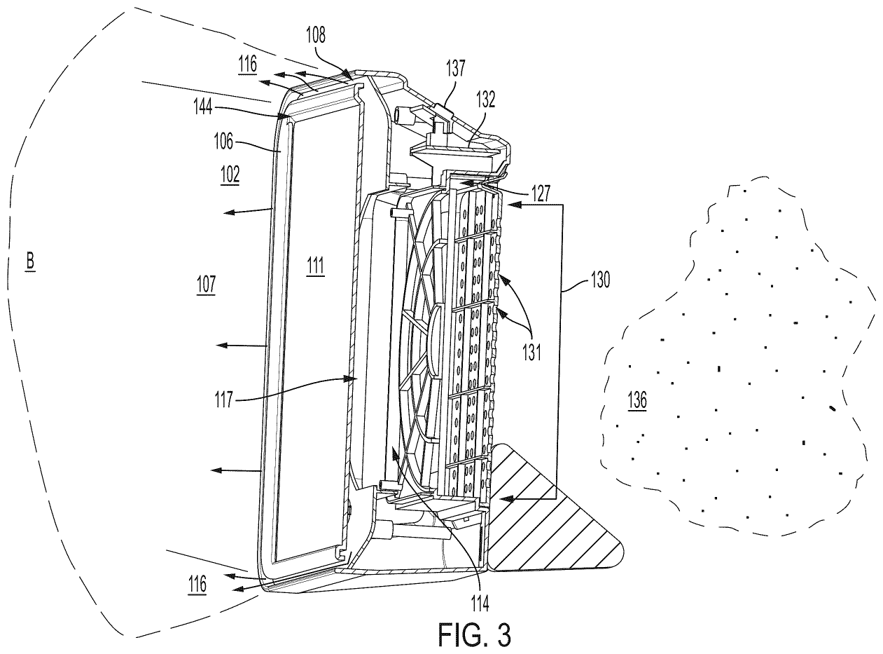

FIG. 3 shows a side cutaway view of the device in FIGS. 1a, 1b, 2a, 2b with the fan 125 and filter 128 removed. Front housing component 102 and rear housing component 103 interface to form main chamber 114. Front baffle 106 covers a front opening 117 and in concert with front housing component 102 creates air discharge region 107 comprising exit port 108. An element for increasing the air pressure, such as fan 125 (not shown) increases the air pressure in the main chamber 114 while simultaneously decreasing the air pressure on the interior of rear chamber 127. This causes ambient 336 to be drawn into the device through air intake region 130 via air inlet ports 131 and through filter 128 (not shown), located in rear chamber 127. The element for increasing air pressure compresses the air in the main chamber 114 for creating the outward flowing air 116, which exits through exit port(s) 108. Control circuit board assembly 132 includes one or more user controls 137 for controlling operation of the system.

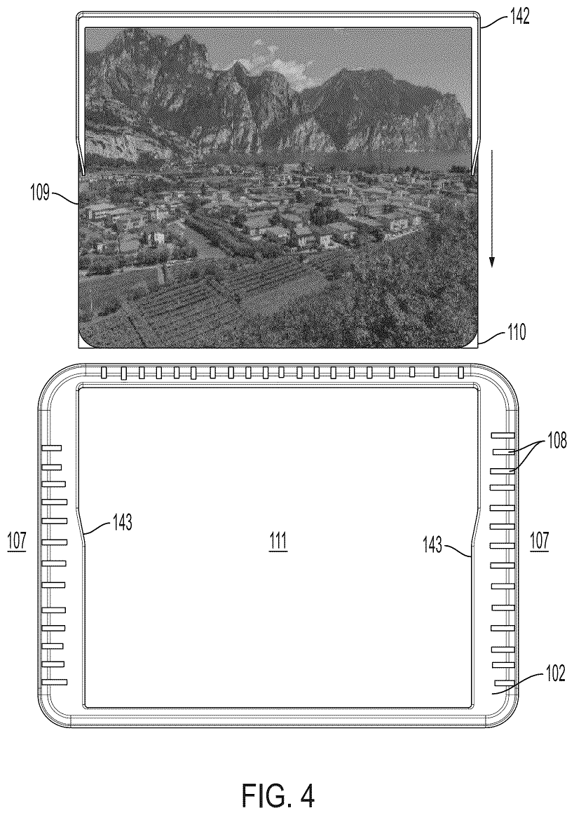

FIG. 4 illustrates an alternate embodiment of an air purifier device where exit ports 108 are formed integral with front housing component 102 such that some portion of front housing component 102 forms air discharge region 107. Display region 111 is also formed integral with front housing component 102. Display region 111 is configured to retain a visual element 109, such as a photograph, via front holding features 141. Frame element 142 may be used to further secure visual element 109 and is configured to engage with front holding features 141. Front holding features may also secure optional clear pane 110 in front of visual element 109. Frame element 142 may be secured to, or formed integral with, clear pane 110. Front holding features 141 comprise slots with a wider opening at one end to allow insertion of a visual element, which may be inserted behind front holding features 141. In an embodiment, frame element 142 engages with front holding features 141 such that the visual element is framed around its perimeter by the combination of front holding features 141 and frame element 142.

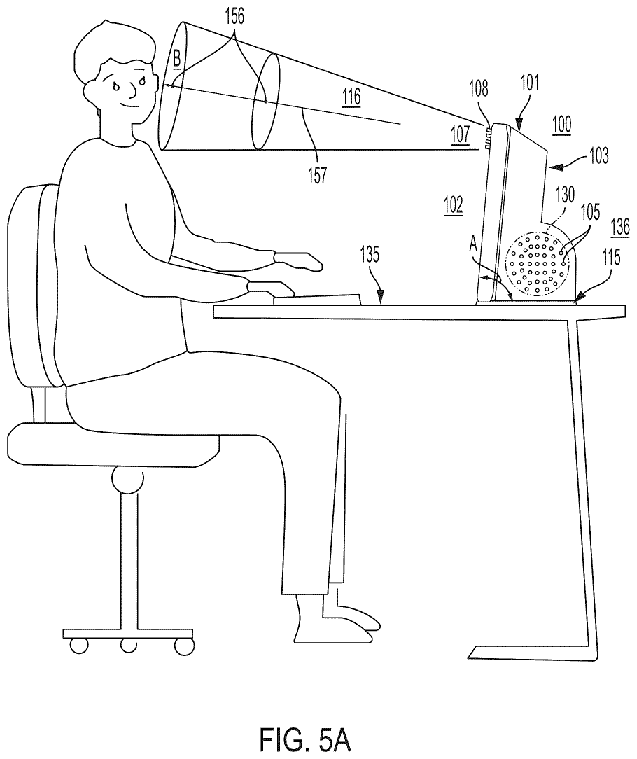

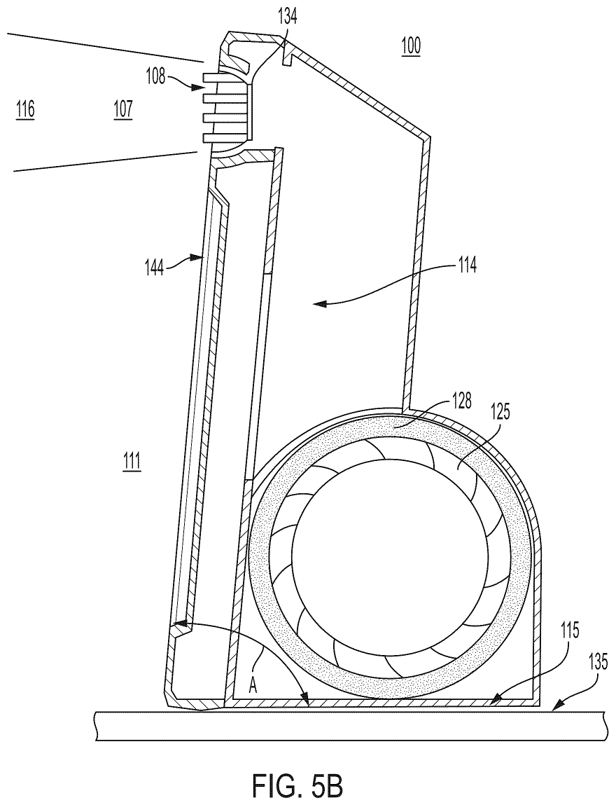

FIG. 5A and FIG. 5B show a side view and a side cutaway view respectively of an air purifying system according to one embodiment. Air purifying device 100 includes housing 101 having a front 102, rear 103 and bottom 115. Bottom 115 is configured to rest on a solid surface 135 such as a desk, table, etc. Front 102 forms an acute angle A with bottom 115 so that outward flowing air 116 from air discharge region 107 via exits ports 108 is angled forward and upward relative to bottom 115. Housing 101 includes air intake region 130 with air inlet ports 105 for accepting ambient air 136 into the device. Exit port 108 includes a movable element 134 to enable altering the direction of the outward flowing air 116 relative to the solid surface. Housing 101 is configured with an air pressure increasing element 125 for drawing in ambient air 136 through a substantially cylindrical filter element 128 and increasing the air pressure in a main chamber 514 operatively coupled to one or more exit port 108. Front 102 includes display region 111.

As outward flowing air 116 is discharged from air discharge region 107 via exit port(s) 108 toward a user in a user position, the outward flowing air 116 creates a stream of moving air which expands to form a wave front B with an increasingly larger cross-sectional area at any distance from device 100, as the stream of air moves away from device 100. The geometric center 156 at every distance away from device 100 forms an imaginary line or vector 157 representing the primary direction of outward flowing air 116. The primary direction of air flow 116 may described by the angle of the imaginary line relative to the surface 135 upon which device 135 is resting upon.

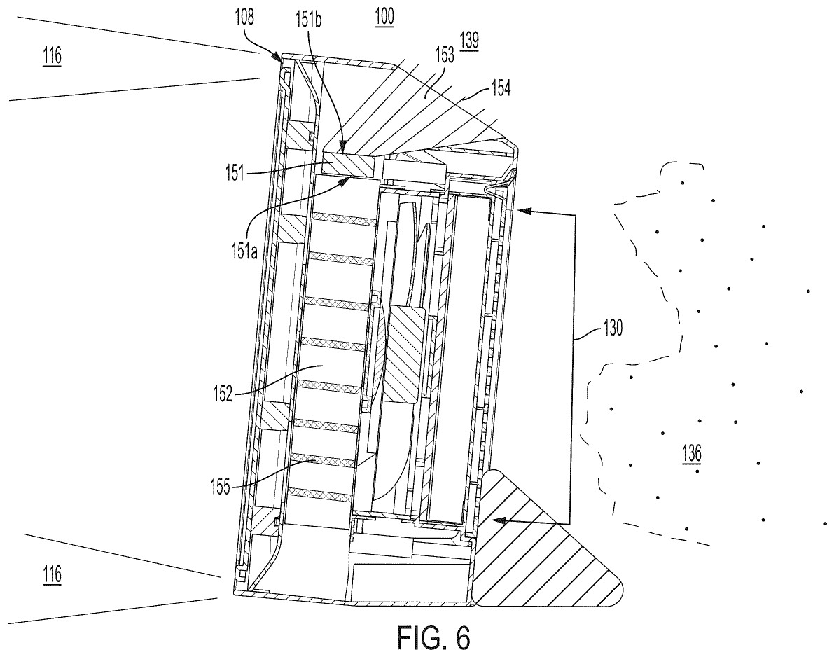

FIG. 6 illustrates an air purifying device according to an embodiment with a condition altering element 151 for altering a property of the outward flowing air 116 relative to the ambient air 136. Condition altering element 151 may alter the temperature, humidity, smell, or other properties of air moving through the device 100 before the air is moved through exit ports 108. Condition altering element 151 may comprise a thermo-electric heat pump, such as a Peltier device which can move heat from one side to another. For example, with the conditional altering element 151 arranged so a first side 151a is toward the interior of device 100 and a second opposing side 151b toward the exterior of device 100, the first side 151a may be made warm by passing current through condition altering element 151, while the second opposing side 151b is made cold. Various thermal elements 152, 153 in good mechanical and thermal contact may be used to create expanded surface areas of the respective warm and cold sides. Thermal elements 152, 153 may be made from materials with high thermal conductivity, such as various metals. Thermal element 152 is positioned so that air passing through device 100 passes over a large warm surface area, thereby making the air warmer than ambient air 136 before exiting device 100 through exit port 108. The surface area of thermal element 152 may be increase with addition of vane features 155. Thermal element 153 is positioned so that the cold surface area of thermal element 153 extends through the housing of device 100 and is brought into contact with ambient air 136. The amount of surface area in contact with ambient air may be increased with vane features 154. Alternatively, by passing current through the condition altering element 151 in the opposite polarity, the side toward the interior of device 100 may be made cold such that air passing through device 100 is made colder than the ambient air 136 entering through air intake region 130 before exiting device 100 through exit port 108 and heat on the exterior side may be conducted out of the housing of device 100 via thermal element 153. The location of thermal element 153 relative to air intake region 130 minimizes the effect of localized ambient air 139 in the region surrounding thermal element 153 on ambient air 136 near air intake region 130.

In an embodiment, condition altering element 151 comprises a heating element configured such that air flowing through device 100 passes over condition altering element 151 and/or any thermal elements 152. In this manner air is warmed by condition altering element 151 alone or in combination with any thermal elements 152 before exiting device 100 via exit port(s) 108. In this construction, condition altering element 151 and any related components may be fully enclosed within the housing of device 100 with no need for any thermal element 153 to extend through the housing or be in contact with ambient air 136.

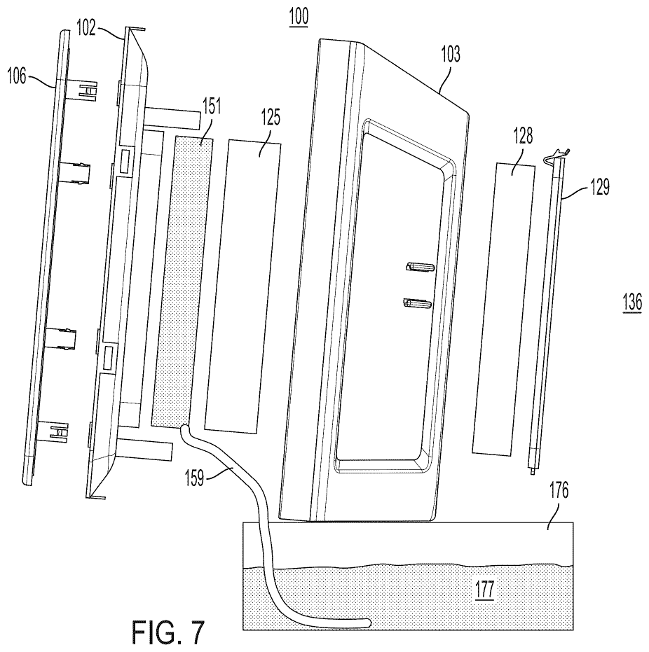

FIG. 7 shows an alternate arrangement of a desktop air purifier system 100 with filter screen 129, filter 128, rear housing component 103, fan 125, front housing component 702 and front baffle 106. A condition altering element 151 is configured to change the relative humidity of air exiting the system 100 relative to ambient air 136 entering system 100. System 100 includes tank 176 capable of holding water 177, which may be drawn from tank 176 via wicking element 159. Tank 176 may be internal, external, or a combination thereof, to the housing of system 100. Tank 176 may be removably engaged with a housing component, for example 103, to allow for easy removal and filling of tank 176 with water 177. With condition altering element 151 extending into the path of air flow through system 100, water 177 drawn from tank 176 into condition altering element 151 via wicking element 159 will be added to air passing over, or through, condition altering element 151 before exiting system 100. In this manner, air exiting system 100 will have higher relative humidity than ambient air 136 entering system 100.

In an alternate arrangement, wicking element 159 is replaced with a hose extending into tank 176 and into water 177. Water 177 is drawn through the hose into condition altering element 151 via a pump (not shown).

Condition altering elements described above, and configured to modify some attribute of the air passing through the device (100, 100), may be included individually or in any combination.

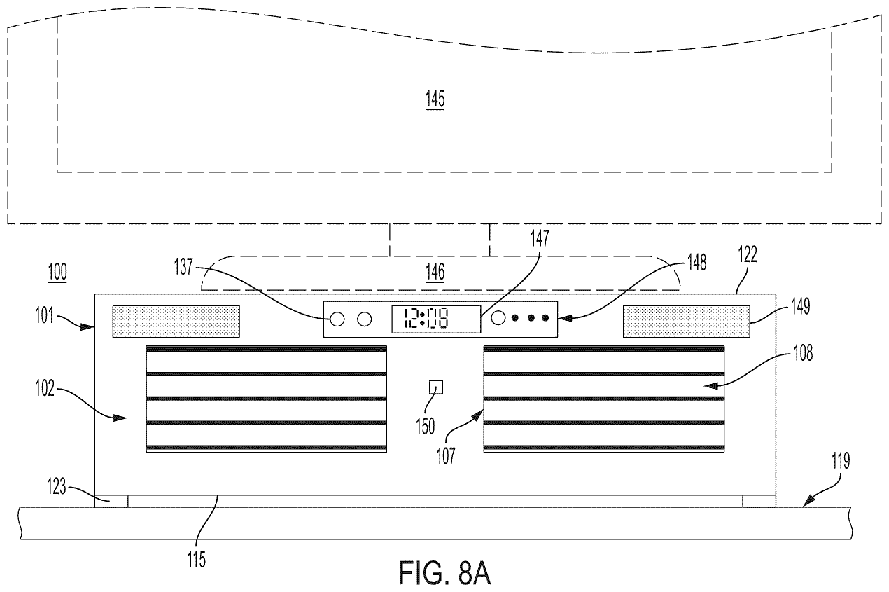

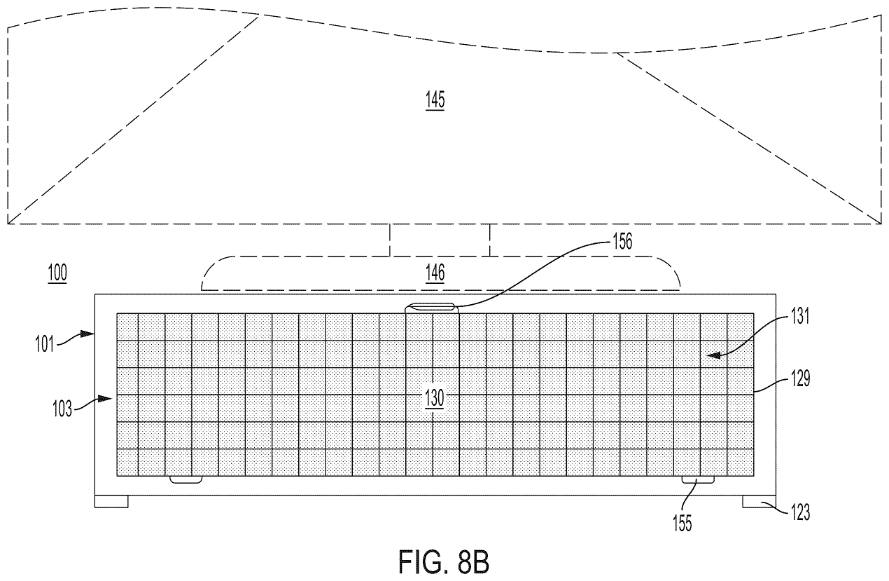

FIGS. 8A and 8B show a front and rear view, respectively, of an embodiment of the invention configured to support an external device with display, such as a computer monitor, television monitor, laptop or similar product. Device 800 includes housing 101 having a front 102, rear 103, bottom 115 and top side 122, with top side 122 arranged to be substantially parallel to bottom 115. Feet 123 may be used to elevate bottom 115 above a resting surface 119. Top side 122 is constructed to be structurally stable enough to support the external device, such as a computer monitor 145, which may include a base or stand 146. Front 102 includes one or more air discharge region(s) 107 comprising one or more exit port(s) 108 for providing outflowing air and directing the outflowing air toward a user, users, or a target area. Exit port 108 may include one or more movable features 134 to enable variable aiming of the outflowing air to change the angle of the outflowing air relative to resting surface 119. Front 102 may be arranged perpendicular to bottom 115 or arranged to form an acute angle with bottom 115 such that outward flowing air is directed upward relative to resting surface 119.

Device 800 includes user control circuit assembly (not shown) in electrical connection to one or more user interface elements 137 for accepting input from a user or providing feedback to a user. The user interface area 148 may also include a display 147, either numeric or graphic, for visual feedback to a user about the operating status of the system. The user control circuit and user interface area 148 may comprise logic, circuitry and display for performing clock functions such as time, date, timer, calendar, etc, which may be used simply to inform the user or may be used to control operation of the system at predetermined times or intervals. System 800 may also include one or more speaker 149 for audio playback and/or microphone 150 for audio input. Microphone 150 may be configured to provide voice command of operation of device 800. In cases where a speaker and/or microphone is included, system 800 includes audio interface circuitry, which may include wired or wireless audio connection to external devices, as is known in the art.

Rear of system 800 includes an air intake region 130 for accepting ambient air into system 800 via one or more air inlet port 131. Air intake region 130 and air inlet ports 131 may comprise a removable panel 129 or screen secured by engagement members 855, 856 which may comprise catches, latches, hinges, tabs, knobs, screws, or other engageable elements as appropriate.

FIG. 9 shows an exploded side view of an embodiment of an air purifier system with included air duct system. The system comprises housing 101, filter 128, filter screen 129 and fan 125. Rear duct 160, having rear duct walls 162, is configured to fit into housing 101 and includes an opening (not shown) aligned with fan 125 when installed. The opening allows air from fan 125 to pass through rear duct 160 toward front panel 102, which includes sloped walls 161 on its rear surface. The system further includes removable front baffle 106. Front panel 102 includes front panel boss 173 which is received into receiver 174 in rear duct 160.

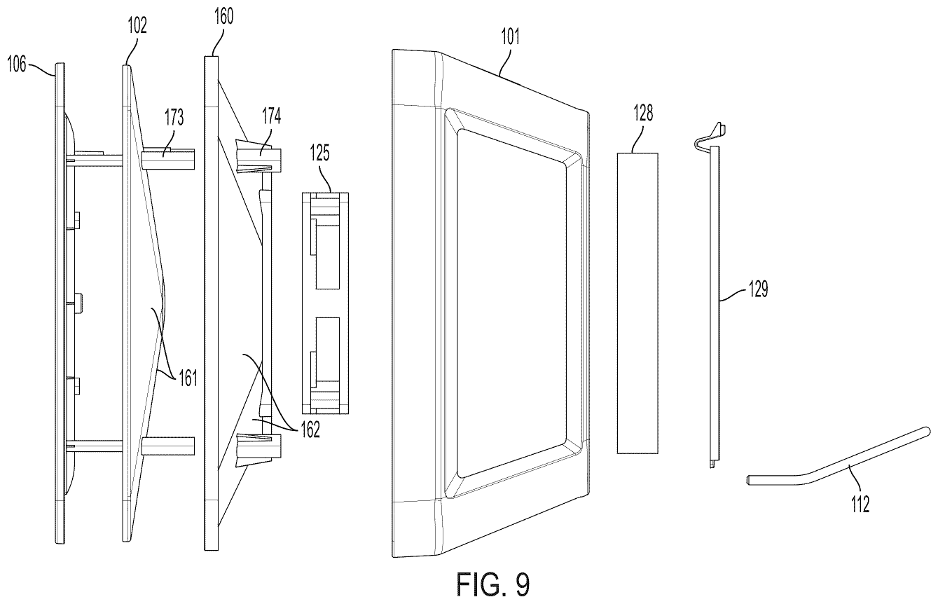

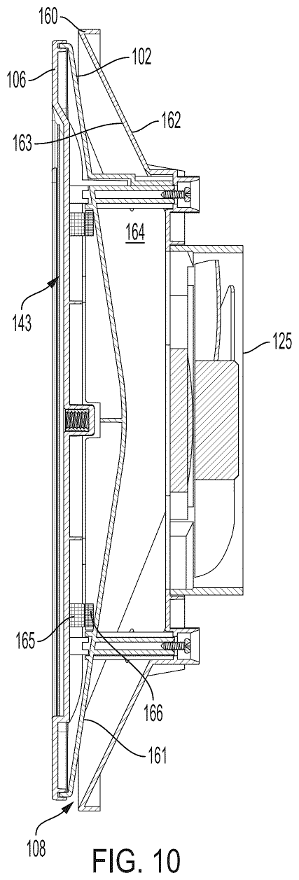

FIG. 10 shows a cutaway side view of a portion of an embodiment of an air purifier system with included air duct comprising rear duct 160 and front housing component 102. The air duct is used to guide air from fan 125 to exit port 108. The air duct is created by the space 164 between the front surface 163 of rear duct wall 162 and the rear surface of front housing component 102, which includes sloped walls 161. Front baffle 106 is removably attached to front housing component 102 using a front securing element 165 which is affixed to or embedded within front baffle 106. Front securing element 165 engages with rear securing element 166 which is affixed to or embedded within front housing component 102. Front securing element 165 and corresponding rear securing element 166 may include hook and loop fasteners, interlocking mechanical components, magnets, magnetic material, tape surfaces, removable glue, latches, tabs, slots, resilient mounting features, screws, rotating locks, clips, or other such components which allow for removable securing of front baffle 106 to front housing component 102 without damage. This configuration allows for easy removal of front baffle 106 for a variety of purposes. For example, front baffle 106 may include provisions for holding a photograph or electronic display (not shown) and removal of front baffle 106 may facilitate the installation or removal of the photograph or electronic display. Additionally, removal of front baffle 106 can provide for replacement of front baffle 106 with an alternate front baffle having other or additional features. This can provide the ability for a system with a variety of secondary uses or upgrades.

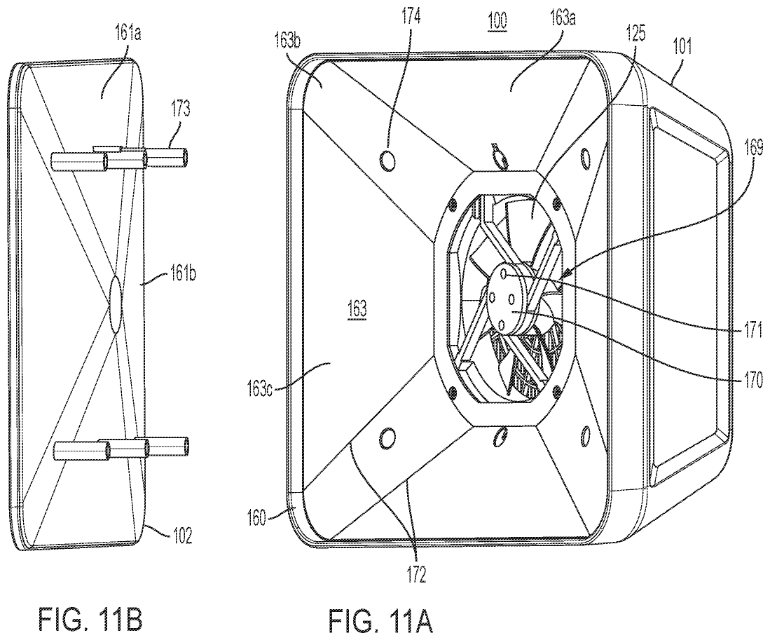

Front surface 163 of rear duct wall 162 and/or rear surface of front housing component 102 may be configured or treated to be reflective to visible light so that internal lighting 133 or 171, shown in FIG. 2A and FIG. 11A respectively, will be directed toward exit port 108 and visible from outside the system. One or more rear duct walls 162, or the entire structure of rear duct 160, may be constructed of thermally conductive material. so that front surface 163, or a portion of front surface 163, acts as thermal element 152 shown in FIG. 6. In this manner, air passing over front surface 163 may be conditioned by conditioning element 151.

FIGS. 11A and 11B show a separated view of various elements of an embodiment of an air purifier system with included air duct comprising rear duct 160 and front housing component 102, which may be assembled using one or more front panel boss 173 each being received into a corresponding receiver 174 and secured from the rear of housing 101 using screws (not shown). Front housing component 102 may be secured to rear housing 101 via other means such as screws extending through front panel bosses 173 and into corresponding receivers in housing 101 or rear duct 160. Rear duct 160 includes duct opening 169 aligned with fan 125 to permit air from fan 125 to pass through rear duct 160 toward sloped walls 161a, 161b of front housing component 102. When assembled, sloped walls 161a, 161b in combination with surface or faces 162a, 162b, 162c of rear duct 160 direct air from fan 125 toward the perimeter of front housing component 102. FIG. 11A shows a rear assembly 100 comprising housing 101, rear duct 160, and fan 125. Rear assembly 100 further includes one or more lighting elements 171, such as a light emitting diode (LED), which may be installed directly into features of rear assembly 100 or may be assembled on a printed circuit board (PCB) 170 which is then installed in rear assembly 100. Front surface 163 of rear duct 160 may have a continuous smooth surface or may comprise a series of wall segments as indicated by faces 163a, 163b, and 163c, which meet to create corners or angles 172.

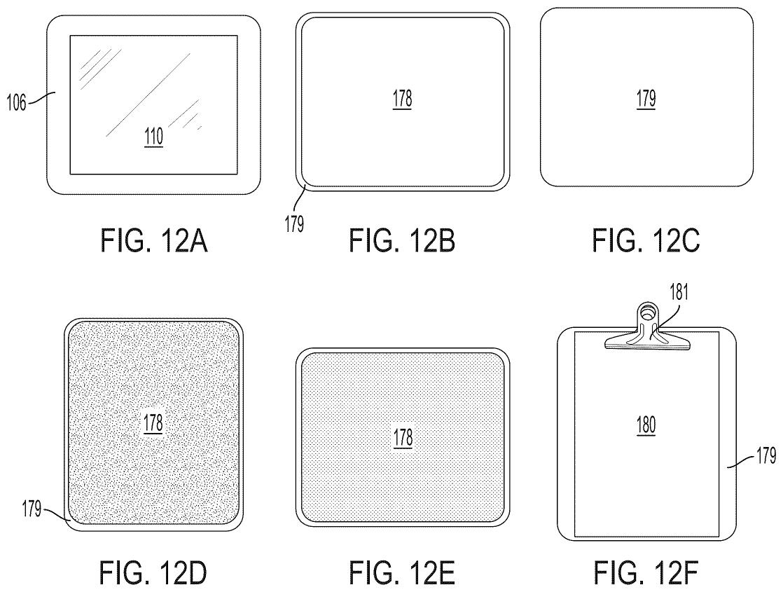

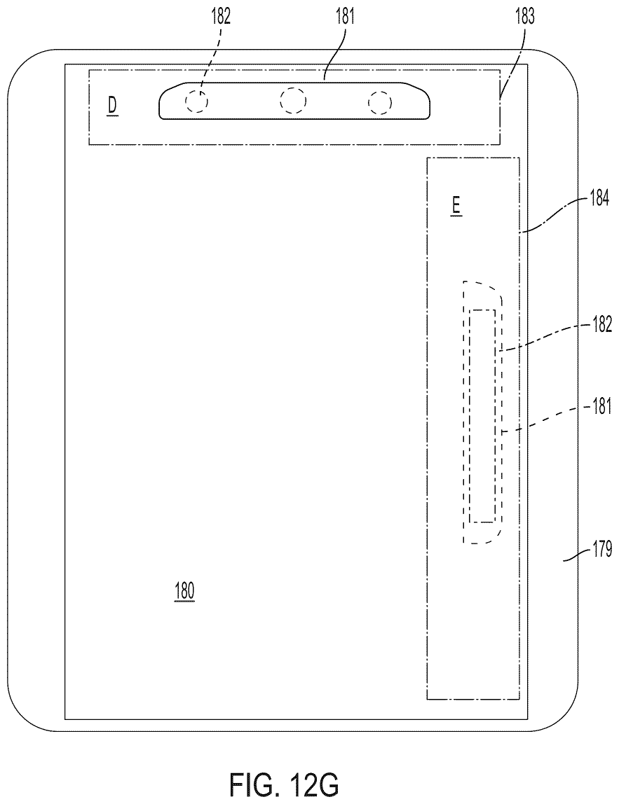

FIG. 12A, 12B, 12C, 12D, 12E, 12F show various configurations of front baffle 106 including functional elements, comprising various constructions, materials and functional surface treatments. FIG. 12A shows a version of front baffle 106 configured to secure a photograph or other visual element, which may be secured behind translucent or transparent panel 110. Front baffle 106 may have a panel surface 179 that comprises a single continuous surface material, which may be the same material and integral with front baffle 106, as shown in FIG. 12C, or may comprise a functional surface treatment 178 over a portion of front surface 179 of front baffle 106 as shown in FIGS. 12B, 12D and 12E. Functional surface treatments 178 may comprise a hard surface such as a dry erase surface shown in FIG. 12B or chalk board or similar surface for writing upon, and may further comprise a magnetic material for use with removable magnets for securing items to the hard surface. Functional hard surface treatments 178 may also be configured for accepting removable tape, self-adhesive tabs or notes, or other such stationary items. Functional surface treatment 178 may comprise a soft surface such as a corkboard as shown in FIG. 12D, fabric as shown in FIG. 12E, or any of a variety of other soft materials which could include the function of accepting pushpins, tacks or other pins for securing items to front baffle 106.

FIG. 12F shows a configuration of front baffle 106 comprising an item retaining feature 181, which may comprise a clip, clamp, bracket, sleeve, magnet, or other feature configured to secure a document 180, visual element, card, envelope, calendar, note paper, etc to front baffle 106.

FIG. 12G shows another embodiment of front baffle 106 configured to secure a document or other visual element. Item retaining feature 181 is configured to secure to panel surface 179 via one or more magnet(s) 182 attached to or embedded within item retaining feature 181. Panel surface 179 may comprise a magnetic material across the entirety of the surface, or integrated into front baffle 106, or may include magnetic material in select magnet regions 183, 184. The attraction of magnet(s) 182 to the magnetic material in panel surface 179 or one or more magnetic regions 183, 184 of panel surface 179 is sufficient to hold document 180 or other items. Item retaining feature 181 may be removed and replaced at any point on panel surface 179 having magnetic material, such as movement from position ‘A’ in magnetic region 183 to position ‘B’ in magnetic region 184 or any other position having a magnetic region. This allows easy transition and configuration when using the air purifying system in either portrait or landscape orientation. In an alternative configuration, front baffle 106 comprises one or more magnets and item retaining feature 181 comprises magnetic material which is secured to front baffle 106 by magnetic attraction, thereby securing document 180.

Each of the various front baffle 106 configurations shown and described above may be employed singly, or front baffle 106 may be made removable to allow the user to interchange two or more of the various front baffle 106 configurations.

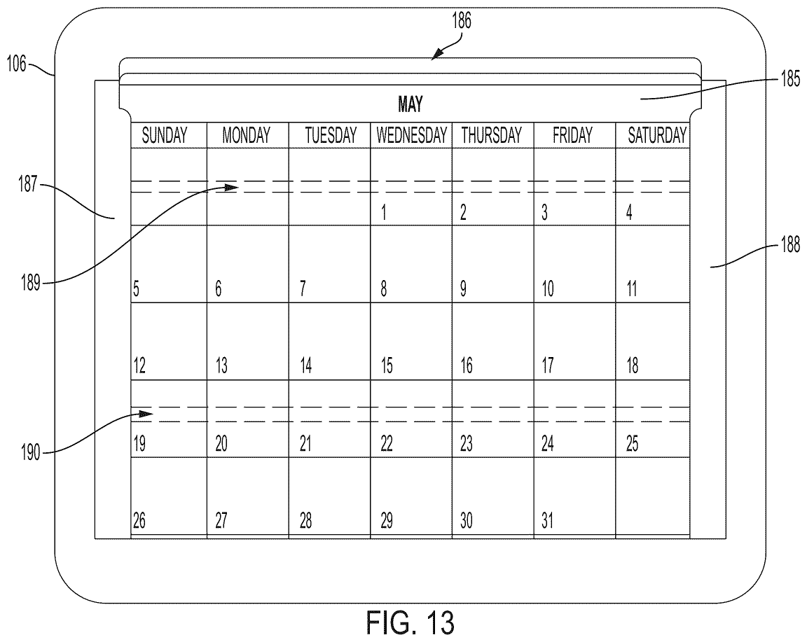

FIG. 13 shows another embodiment of front baffle 106 configured with a slot 186 including a space between two retaining sides 187, 188 for accepting and holding an item, such as calendar 185. The slot 186 is may be configured for holding an item according to a standardized size, such as a piece of 8.5″×11″ paper, 8″×10″ photograph, 5″×7″ photograph, etc. The size of the slot and the open dimension between the retaining sides may be adjusted or made adjustable to accommodate the size of the standardized item to be held. For example, retaining sides 187, 188, or portions of retaining sides 187, 188, made be constructed to slide along channels 189, 190 in front baffle 106 to vary the opening between retaining sides 187, 188.

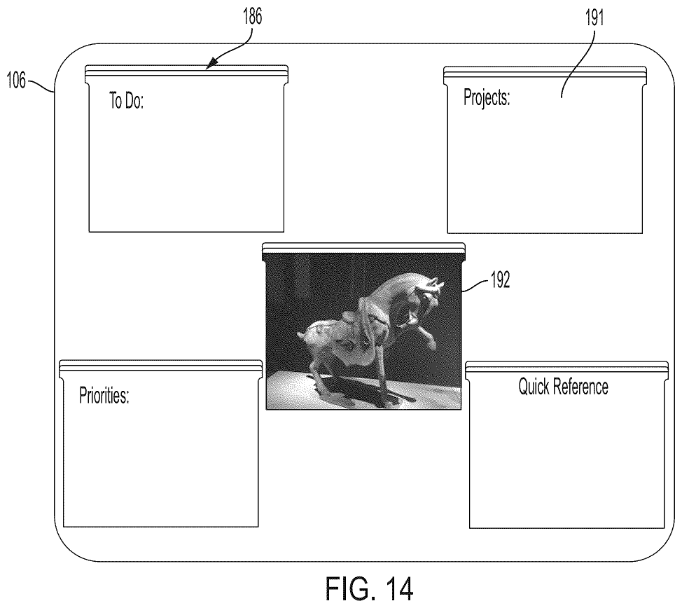

FIG. 14 illustrates an embodiment where the front panel 106 incorporates multiple pockets with slots 186 to retain multiple items 191, such as notes, cards, photographs 192, or other items for easy viewing.

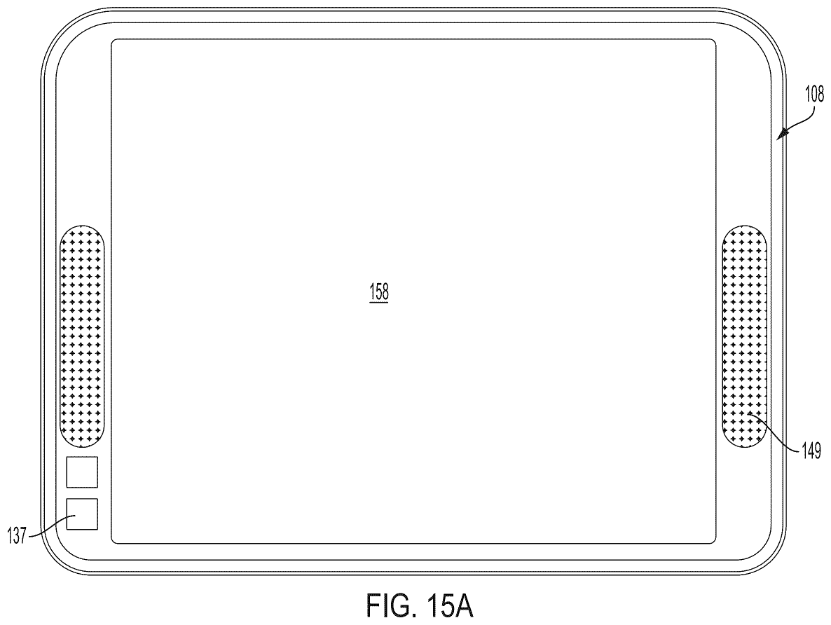

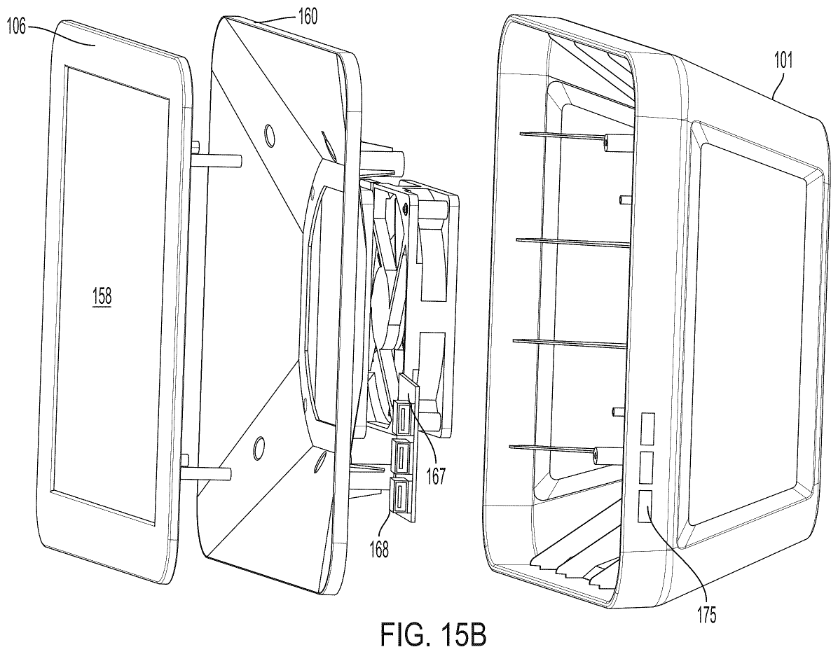

FIGS. 15A and 15B illustrates an embodiment of an air purifier system with electronic display 158 integral with front baffle 106 and provision for connection to external devices. User interface controls 137 enable independent or simultaneous operation of the electronic display 158 and the air purifier system. The system further includes one or more speaker 149 to enable audio playback in combination with electronic display 158 or independently. Exit port 108 surrounds the perimeter of electronic display 158. The system includes circuit board 167 which includes circuitry to provide charging capability over connectors 168. Circuit board 167, may optionally provide data communication between the air purifier system and an external device, such as a computer. When the system is assembled, connectors 168 are accessible through connector openings 175 in housing 101. Connectors 168 may include universal serial bus (USB) connectors for delivery of power to external devices. Further, circuit board 167 may include appropriate circuitry to enable the system to function as a USB hub. Data exchanged over connectors 168 may be used to control system operation, provide content for display on electronic display 158 or combinations thereof.

I claim:

1. An air purifying device comprising:
a housing configured to rest on a surface, the housing having a rear and a front;
an air pressure altering element located within the housing;
a filter assembly configured to remove contaminants from air as air is passed through the filter assembly;
an air intake region comprising an intake port;
a chamber interior to the air intake region and configured to accept the filter assembly such that the filter assembly is located between the intake port and the air pressure altering element;
a main chamber operatively coupled to the air pressure altering element whereby the air pressure altering element increases air pressure in the main chamber, the main chamber further operatively coupled to an exit port on the front;
an air discharge region comprising at least the exit port and configured so that air leaving the device forms a tapered, conical or wedge-shaped stream of moving air having a smaller cross-sectional area at the device and spreading out to increasingly larger cross-sectional area as distance from the device increases;
and wherein the device is configured so that outward flowing air leaves the device through the exit port at an upward angle relative to the surface.

2. The device of claim 1 wherein the device is configured such that the stream of moving air is directed toward a user in a user position and wherein the stream of moving air arriving at the user position is configured to fully encompass the user's head.

3. The device of claim 2 wherein flow of air through the device creates purified air and wherein the stream of moving air comprises purified air and wherein the stream of moving air arriving at the user position forces ambient air away from the user and creates a condition whereby the user breathes substantially all purified air.

4. The device of claim 2 wherein the stream of moving air has a cross-sectional area of at least seventy-five square inches at a distance of eighteen inches from the exit port.

5. The device of claim 1 wherein a majority of the front is configured to hold a photograph and wherein the air discharge region is formed peripheral to the photograph.

6. The device of claim 1 wherein a majority of the front comprises an electronic display capable of displaying graphics, images, videos, or combinations thereof and wherein the air discharge region is formed peripheral to the electronic display.

7. The device of claim 6 further comprising a link to an external device or system and wherein the electronic display is configured to display information, content, or images received from the external device or system.

8. The device of claim 1 further comprising an element configured to alter a condition of outward flowing air relative to ambient air, the element configured to alter a thermal condition, a humidity condition, ionic condition, or combinations thereof.

9. The device of claim 8 wherein the element is a thermal element configured such that air exiting the device is at a different temperature than ambient air entering the device.

10. An air purifying device comprising:
a housing configured to rest on a surface, the housing having a rear and a front;
an air pressure altering element located within the housing;
a filter assembly configured to remove contaminants from air as air is passed through the filter assembly;
an air intake region comprising an intake port;
a chamber interior to the air intake region and configured to accept the filter assembly such that the filter assembly is located between the intake port and the air pressure altering element;
a main chamber operatively coupled to the air pressure altering element whereby the air pressure altering element increases air pressure in the main chamber, the main chamber further operatively coupled to an exit port on the front;
a rear duct within the housing configured between the air pressure altering element and the front, the rear duct having a front surface, the rear duct further having an opening to allow air from the air pressure altering element to pass toward the front surface;
a front housing component having a rear surface with sloped sides, the front housing component configured in front of the rear duct such that the rear surface of the front housing component and the front surface of the rear duct form a passageway to direct air from the air pressure altering element toward the exit port arranged along at least a portion of a perimeter of the front;
an air discharge region comprising at least the exit port;
and wherein the device is configured so that outward flowing air leaves the device through the exit port at an upward angle relative to the surface.

11. The device of claim 10 wherein the front has a display region.

12. The device of claim 11 wherein the air discharge region is peripheral to the display region.

13. The device of claim 10 wherein at least a portion of the front surface of the rear duct comprises a thermally conductive material.

14. A device for improving a user's local environment, the device comprising:
a housing, the housing having a top, a first side configured to rest on a surface in a first orientation, a second side, a rear and a front;
an air pressure altering element located within the housing;
a filter assembly configured to remove contaminants from air as air is passed through the filter assembly;
an air intake region comprising an intake port;
a main chamber operatively coupled to the air pressure altering element whereby the air pressure altering element increases air pressure in the main chamber, the main chamber further operatively coupled to an exit port on the front;
an air discharge region comprising at least the exit port and configured so that air leaving the device forms a tapered, conical or wedge-shaped stream of moving air having a smaller cross-sectional area at the device and spreading out to increasingly larger cross-sectional area as distance from the device increases, the stream of moving air having a geometric center representing a primary direction;
and wherein the device is configured so that the stream of moving air exits the front of the device through the exit port and forms an angle between the geometric center and the surface.

15. The device of claim 14 wherein the primary direction is substantially perpendicular to the front.

16. The device of claim 15 wherein said angle is between 5° and 60°.

17. The device of claim 15 wherein the angle is adjustable via a movable element.

18. The device of claim 15 wherein the second side is also configured to rest on the surface in a second orientation such that the angle is determined by an angle of intersection between the front and the second side.

19. The device of claim 15 wherein the device is configured such that the stream of moving air is directed toward a user's head in a user position and wherein the stream of moving air at the user position is configured to fully encompass the user's head.

20. The device of claim 14 further comprising an audio system including at least one speaker or at least one microphone.

21. The device of claim 14 configured as a desktop display system wherein the front comprises a front baffle extending across a majority the front and wherein said front baffle includes a display region extending across a majority of the front baffle, and wherein the air discharge region is peripheral to the display region.

22. The device of claim 21 wherein the display region is configured to hold a photograph.

23. The device of claim 21 wherein the display region comprises an electronic display capable of displaying graphics, images, videos, or combinations thereof.

24. The device of claim 23 further comprising a link for connection to an external electronic device or system and the electronic display is configured to display content based on data received from the external device or system via the link.

25. The device of claim 24 wherein the link comprises a connector.

26. The device of claim 14 further comprising a front baffle configured to secure an external electronic, the external device having an integral electronic display.

27. The device of claim 14 further comprising a front baffle having one or more functional elements.

28. The device of claim 27 wherein the front baffle is removable.
